(19) United States
(12) Patent Application Publication
(10) Pub. No.: US 2026/0232254 A1
(43) Pub. Date: Aug. 13, 2026

(54) ELECTROENCEPHALOGRAPHY ELECTRODE PLACEMENT FOR BRAIN-COMPUTER INTERFACE APPLICATIONS

(71) Applicant: University of Florida Research Foundation, Incorporated, Gainesville, FL (US)

(72) Inventor: Nicholas Rodriguez, Gainesville, FL (US)

(21) Appl. No.: 19/537,807

(22) Filed: Feb. 12, 2026

Related U.S. Application Data
(60) Provisional application No. 63/757,905, filed on Feb. 13, 2025.

(51) Int. Cl. A61B 5/384; A61B 5/386

(52) U.S. Cl. CPC A61B 5/384; A61B 5/386

(57) ABSTRACT

Various embodiments of the present disclosure provide methods, apparatus, systems, computing devices, computing entities, and/or the like for receiving, by one or more processors, electroencephalography (EEG) data and one or more current electrode positions from an EEG device; determining a subject EEG task corresponding to a current state of the EEG device; generating a latent space representation corresponding to the EEG data and the one or more current electrode positions; generating an uncertainty measure corresponding to the latent space representation and the EEG data; generating one or more preferred electrode positions corresponding to the subject EEG task and the EEG device; and generating a visual representation of the one or more preferred electrode positions.

### CROSS-REFERENCES TO RELATED APPLICATION(S)

The present application claims priority to U.S. Patent Application No. 63/757,905 (filed on February 13, 2025), which is incorporated by reference herein in its entirety.

### BACKGROUND

Various embodiments of the present disclosure address technical challenges related to electroencephalography electrode placement in brain computer interface applications.

Electroencephalography (EEG) is a procedure by which electrical activity within a brain is measured to help diagnose or detect a variety of conditions. Cells within the brain communicate via electrical impulses which are detectable using small metal discs called electrodes which are placed on the scalp. During an EEG, brain wave patterns are recorded using the electrodes to produce an EEG recording depicting a plurality of wavy lines. Normal brain activity conforms to a recognizable pattern within the EEG recording, while abnormal patterns may be an indication of a condition or other problem.

### BRIEF SUMMARY

In general, various embodiments of the present disclosure provide methods, apparatus, systems, computing devices, computing entities, and/or the like for determining electrode placement for passive brain-computer interface applications.

In some embodiments, a computer-implemented method comprises: receiving, by one or more processors, electroencephalography (EEG) data and one or more current electrode positions from an EEG device; determining, by the one or more processors, a subject EEG task corresponding to a current state of the EEG device; generating, by the one or more processors, a latent space representation corresponding to the EEG data and the one or more current electrode positions; determining, by the one or more processors, an uncertainty measure corresponding to the latent space representation and the EEG data; determining, by the one or more processors, one or more preferred electrode positions corresponding to the subject EEG task and the EEG device; and generating, by the one or more processors, a visual representation of the one or more preferred electrode positions.

In some embodiments, a computing apparatus comprises memory and one or more processors communicatively coupled to the memory, the one or more processors configured to perform operations comprising: receiving electroencephalography (EEG) data and one or more current electrode positions from an EEG device; determining a subject EEG task corresponding to a current state of the EEG device; generating a latent space representation corresponding to the EEG data and the one or more current electrode positions; determining an uncertainty measure corresponding to the latent space representation and the EEG data; determining one or more preferred electrode positions corresponding to the subject EEG task and the EEG device; and generating a visual representation of the one or more preferred electrode positions.

In some embodiments, one or more non-transitory computer-readable storage media including instructions that, when executed by one or more processors, cause the one or more processors to perform operations comprising: receiving electroencephalography (EEG) data and one or more current electrode positions from an EEG device; determining a subject EEG task corresponding to a current state of the EEG device; generating a latent space representation corresponding to the EEG data and the one or more current electrode positions; determining an uncertainty measure corresponding to the latent space representation and the EEG data; determining one or more preferred electrode positions corresponding to the subject EEG task and the EEG device; and generating a visual representation of the one or more preferred electrode positions.

### BRIEF DESCRIPTION OF THE DRAWINGS

FIG. 1 provides an example overview of an architecture in accordance with some embodiments of the present disclosure.

FIG. 2 provides an example electrode placement computing entity in accordance with some embodiments of the present disclosure.

FIG. 3 provides an example user computing entity in accordance with some embodiments of the present disclosure.

FIG. 4 provides a dataflow diagram in accordance with some embodiments of the present disclosure.

FIG. 5 depicts an example process executed across a plurality of components in accordance with some embodiments of the present disclosure.

FIG. 6 is a flowchart diagram of an example process for determining electrode placement locations in accordance with some embodiments of the present disclosure.

### DETAILED DESCRIPTION

Various embodiments of the present disclosure are described more fully hereinafter with reference to the accompanying drawings, in which some, but not all embodiments of the present disclosure are shown. Indeed, the present disclosure may be embodied in many different forms and should not be construed as limited to the embodiments set forth herein; rather, these embodiments are provided so that this disclosure will satisfy applicable legal requirements. The term “or” is used herein in both the alternative and conjunctive sense, unless otherwise indicated. The terms “illustrative” and “example” are used to be examples with no indication of quality level. Terms such as “computing,” “determining,” “generating,” and/or similar words are used herein interchangeably to refer to the creation, modification, or identification of data. Further, “based on,” “based at least in part on,” “based at least on,” “based upon,” and/or similar words are used herein interchangeably in an open-ended manner such that they do not necessarily indicate being based only on or based solely on the referenced element or elements unless so indicated. Like numbers refer to like elements throughout.

Embodiments of the present disclosure include systems, methods, computer program products, and techniques for optimizing EEG electrode placement in brain-computer interface (BCI) applications. Such EEG electrode placement optimization techniques integrate several advanced components to achieve high performance with minimal hardware by employing a variational autoencoder (VAE) coupled with uncertainty measure techniques to provide a refined optimization process. This combination allows for efficient exploration of the relationship between electrode placement and signal quality, even with a limited number of electrodes. Some embodiments of the present disclosure are configured to run on specialized hardware, such as neuromorphic chips or GPUs, thereby enabling real-time processing and feedback. Some embodiments of the present disclosure incorporate a graphical user interface (GUI) facilitating intuitive interaction and visualization of the optimization process.

### Computer Program Products, Methods, and Computing Entities

Embodiments of the present disclosure may be implemented in various ways, including as computer program products that comprise articles of manufacture. Such computer program products may include one or more software components including, for example, software objects, methods, data structures, or the like. A software component may be coded in any of a variety of programming languages. An illustrative programming language may be a lower-level programming language such as an assembly language associated with a particular hardware architecture and/or operating system platform. A software component comprising assembly language instructions may require conversion into executable machine code by an assembler prior to execution by the hardware architecture and/or platform. Another example programming language may be a higher-level programming language that may be portable across multiple architectures. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query or search language, and/or a report writing language. In one or more example embodiments, a software component comprising instructions in one of the foregoing examples of programming languages may be executed directly by an operating system or other software component without having to be first transformed into another form. A software component may be stored as a file or other data storage construct. Software components of a similar type or functionally related may be stored together such as, for example, in a particular directory, folder, or library. Software components may be static (e.g., pre-established or fixed) or dynamic (e.g., created or modified at the time of execution).

A computer program product may include a non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media (including volatile and non-volatile media).

In some embodiments, a non-volatile computer-readable storage medium may include a floppy disk, flexible disk, hard disk, solid-state storage (SSS) (e.g., a solid state drive (SSD), solid state card (SSC), solid state module (SSM), enterprise flash drive, magnetic tape, or any other non-transitory magnetic medium, and/or the like. A non-volatile computer-readable storage medium may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile computer-readable storage medium may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

In some embodiments, a volatile computer-readable storage medium may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media may be substituted for or used in addition to the computer-readable storage media described above.

As should be appreciated, various embodiments of the present disclosure may also be implemented as methods, apparatus, systems, computing devices, computing entities, and/or the like. As such, embodiments of the present disclosure may take the form of an apparatus, system, computing device, computing entity, and/or the like executing instructions stored on a computer-readable storage medium to perform certain steps or operations. Thus, embodiments of the present disclosure may also take the form of an entirely hardware embodiment, an entirely computer program product embodiment, and/or an embodiment that comprises combination of computer program products and hardware performing certain steps or operations.

Embodiments of the present disclosure are described below with reference to block diagrams and flowchart illustrations. Thus, it should be understood that each block of the block diagrams and flowchart illustrations may be implemented in the form of a computer program product, an entirely hardware embodiment, a combination of hardware and computer program products, and/or apparatus, systems, computing devices, computing entities, and/or the like carrying out instructions, operations, steps, and similar words used interchangeably (e.g., the executable instructions, instructions for execution, program code, and/or the like) on a computer-readable storage medium for execution. For example, retrieval, loading, and execution of code may be performed sequentially such that one instruction is retrieved, loaded, and executed at a time. In some example embodiments, retrieval, loading, and/or execution may be performed in parallel such that multiple instructions are retrieved, loaded, and/or executed together. Thus, such embodiments can produce specifically-configured machines performing the steps or operations specified in the block diagrams and flowchart illustrations. Accordingly, the block diagrams and flowchart illustrations support various combinations of embodiments for performing the specified instructions, operations, or steps.

### Example Framework

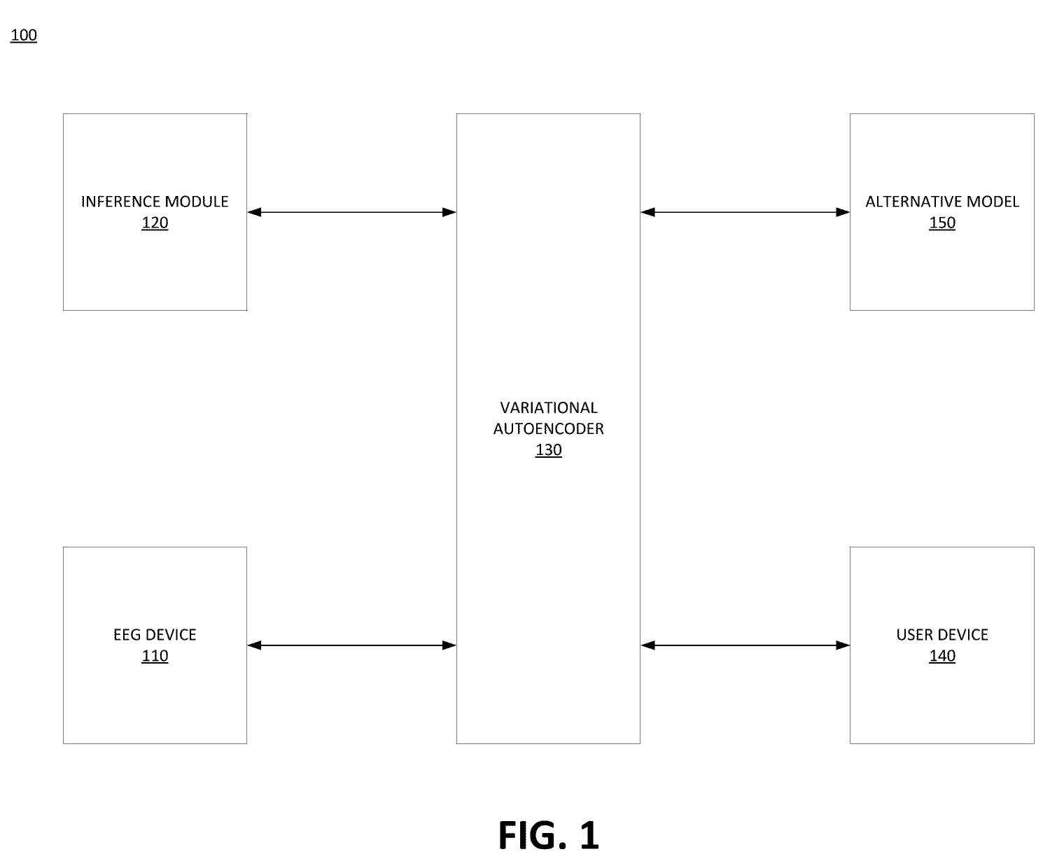

FIG. 1 provides an example overview of an architecture 100 in accordance with some embodiments of the present disclosure. The architecture 100 includes an EEG device 110, an inference module 120, a variational autoencoder 130, a user device 140, and an alternative model 150. Architecture 100 may be configured to provide EEG device calibration and electrode placement optimization. It should be appreciated that, as used herein, “optimized parameters” and other such phrases are used to refer to one or more preferred parameters configured to provide improved performance of the corresponding electrode relative to a current position of said electrode. Similarly, “optimization” of such electrode positions and parameters refers to the process of determining preferred locations for said electrode(s), wherein the preferred locations provide increased performance with respect to the subject electrode(s). Notably, optimization refers to a methodology for improving the electrode placement locations, even in embodiments wherein a truly “optimal” location is not usable (such as those where an injury or other condition prevents utilization of a “best” electrode placement, for example. As such, the terms “preferred parameters” and “optimized parameters” may be used interchangeably herein.

EEG device 110 may include an EEG headset or other device configured to capture raw EEG signals corresponding to an individual. In at least some embodiments, EEG device 110 may include a one or more electrodes or other sensors placed on an individual to monitor the individual’s brain activity. In at least some embodiments, EEG device 110 additionally includes one or more amplifiers. EEG device 110 may additionally be configured to included, or may be operably connected to, a filter. In at least some embodiments, EEG device 110 includes, or is operably connected to, an analog-to-digital converter. In at least some embodiments, such as those wherein EEG device 110 is a portable device, EEG device 110 further includes a battery. EEG device 110 may further include a communications module configured to enable communication with one or more additional devices, such as inference module 120, variational autoencoder 130, user device 140, and alternative model 150. In at least some embodiments, EEG device 110 may be an ambulatory EEG machine used during an extended EEG reading. EEG device 110 may be an EEG neurofeedback machine configured to provide brain activity readings in real time. In at least some embodiments, EEG device 110 may be a video electroencephalogram (VEEG) machine including a camera configured to visually observe the individual while also recording brain activity.

Inference module 120 may be configured to predict the individual’s brain state or features. In at least some embodiments, inference module 120 includes an inference module configured to determine a predicted brain state or features according to one or more optimized parameters. Inference module 120 may be configured to provide feedback to user device 140 and/or variational autoencoder 130. In at least some embodiments, inference module 120 is configured to apply optimized EEG parameters to tasks such as emotional inference or motor intent. For example, inference module 120 may be configured to define optimized parameters for one or more EEG tasks based at least in part on data received from EEG device 110, variational autoencoder 130, and user device 140.

Variational autoencoder 130 may be configured to receive EEG data from EEG device 110. In at least some embodiments, variational autoencoder 130 includes one or more machine learning models configured to generate optimized control parameters corresponding to the EEG device 110. In at least some embodiments, variational autoencoder 130 is configured to produce task-optimized control parameters. Variational autoencoder 130 may additionally be configured to retain latent space representations corresponding to the optimized control parameters. In at least some embodiments, variational autoencoder 130 includes a modified variational autoencoder (VAE) configured for processing the received EEG data. In at least some embodiments, the VAE is a fully spiking VAE. In general, VAE 130 is a machine learning module configured to learn patterns from data by compressing received information into a simplified representation as a latent space. The latent space acts as a map that highlights key features, helping the system focus on the most important data.

In at least some embodiments, such as those where the EEG device 110 is configured to utilize a single electrode or a few electrodes, the variational autoencoder 130 may comprise one or more machine learning models configured to generate optimized control parameters for operation of the EEG device 110 based on the limited electrode availability. For example, when determining optimal placements for a defined number of electrodes, the optimal placement of a selected electrode may be specific to that defined number of electrodes; in other words, an “optimal placement” determined with respect to one or more two electrodes may not necessarily also be an “optimal placement” determined with respect to one or more three electrodes. In some embodiments, the one or more machine learning models may be configured to prioritize or otherwise process certain parameters differently with respect to a limited set of available electrodes; for example, if one or more electrodes is too small to ensure optimal placement with respect to every portion of the brain, the one or more machine learning models may be configured to generate placements which ensure a baseline level of operation and/or which ensure coverage of a selected region of interest, for example.

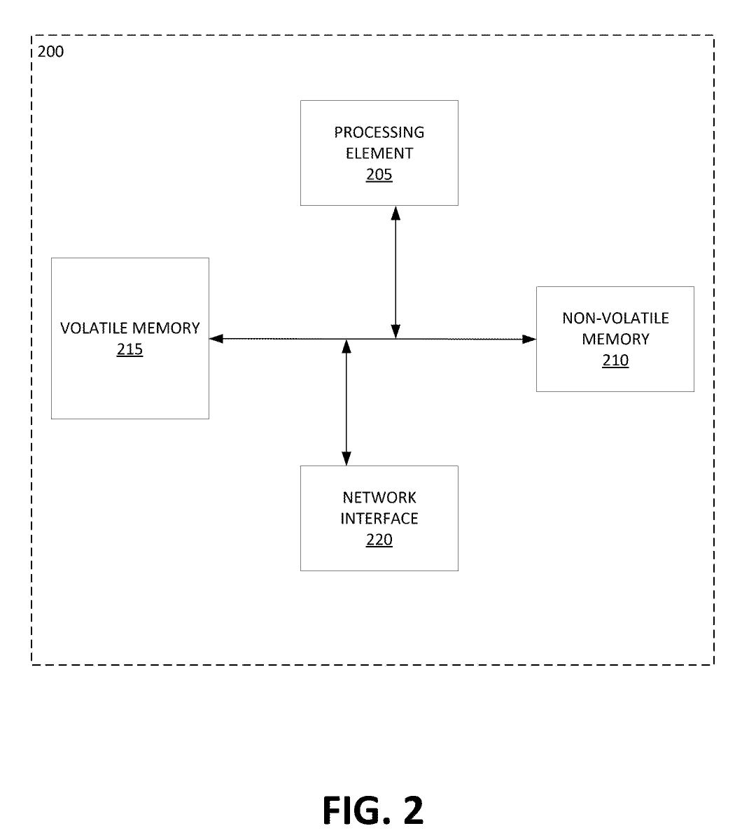

User device 140 may be a laptop, desktop, phone, tablet, system, or other device capable of receiving communications from EEG device 110. In general, user device 140 is representative of any device configured to receive and process data from EEG device 110. User device 140 may include a graphical user interface configured to display data from EEG device 110 to a user. In at least some embodiments, the graphical user interface of user device 140 is further configured to receive user interaction with one or more selectable elements. For example, user device 140 may enable a user to adjust modes and/or parameters of the EEG device 110 (or variational autoencoder 130/inference module 120). In at least some embodiments, user device 140 is configured to display a visual representation of current and/or preferred electrode placement with respect to the EEG device 110. In at least some embodiments, user device 140 is configured with one or more adjustment controls. An example embodiment of a user device 140 is depicted with respect to computing entity 200 of FIG. 2.

Alternative model 150 may be an additional model used in conjunction with the one or more machine learning models of variational autoencoder 130 to provide dual calibration. In at least some embodiments, alternative model 150 may be configured to use supervisory labels (present in the VAE, for example) to provide dual calibration and inference roles. In at least some embodiments, alternative model 150 may be implemented by the VAE of variational autoencoder 130, such that variational autoencoder 130 thereby provides both inference and calibration. In such embodiments, inference module 120 may be configured to update parameters associated with tasks as determined by the variational autoencoder 130.

### Example User Device

FIG. 2 provides an example computing entity 200 in accordance with some embodiments of the present disclosure. The computing entity 200 is an example of the user device 140 of FIG. 1. In general, the terms computing entity, computer, entity, device, system, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein.

As shown in FIG. 2, in some embodiments, the computing entity 200 may include, or be in communication with, one or more processing elements 205 (also referred to as processors, processing circuitry, and/or similar terms used herein interchangeably) that communicate with other elements within the computing entity 200 via a bus, for example. As will be understood, the processing element 205 may be embodied in a number of different ways.

For example, the processing element 205 may be embodied as one or more complex programmable logic devices (CPLDs), microprocessors, multi-core processors, coprocessing entities, application-specific instruction-set processors (ASIPs), microcontrollers, and/or controllers. Further, the processing element 205 may be embodied as one or more other processing devices or circuitry. The term circuitry may refer to an entirely hardware embodiment or a combination of hardware and computer program products. Thus, the processing element 205 may be embodied as integrated circuits, application specific integrated circuits (ASICs), field programmable gate arrays (FPGAs), programmable logic arrays (PLAs), hardware accelerators, another circuitry, and/or the like.

As will therefore be understood, the processing element 205 may be configured for a particular use or configured to execute instructions stored in volatile or non-volatile media or otherwise accessible to the processing element 205. As such, whether configured by hardware or computer program products, or by a combination thereof, the processing element 205 may be capable of performing steps or operations according to embodiments of the present disclosure when configured accordingly.

In some embodiments, the computing entity 200 may further include, or be in communication with, non-volatile media (also referred to as non-volatile storage, memory, memory storage, memory circuitry, and/or similar terms used herein interchangeably). In some embodiments, the non-volatile media may include one or more non-volatile memory 210, including, but not limited to, hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

As will be recognized, the non-volatile media may store databases, database instances, database management systems, data, applications, programs, program modules, scripts, code (e.g., source code, object code, byte code, compiled code, interpreted code, machine code, etc.) that embodies one or more computer functions described herein, executable instructions, and/or the like. The term database, database instance, database management system, and/or similar terms used herein interchangeably, may refer to a collection of records or data that is stored in a computer-readable storage medium using one or more database models; such as a hierarchical database model, network model, relational model, entity-relationship model, object model, document model, semantic model, graph model, and/or the like.

In some embodiments, the computing entity 200 may further include, or be in communication with, volatile media (also referred to as volatile storage, memory, memory storage, memory circuitry, and/or similar terms used herein interchangeably). In some embodiments, the volatile media may also include one or more volatile memory 215, including, but not limited to, RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like.

As will be recognized, the volatile storage or memory media may be used to store at least portions of the databases, database instances, database management systems, data, applications, programs, program modules, code (source code, object code, byte code, compiled code, interpreted code, machine code) that embodies one or more other computer functions described herein, executable instructions, and/or the like being executed by, for example, the processing element 205. Thus, the databases, database instances, database management systems, data, applications, programs, program modules, code (source code, object code, byte code, compiled code, interpreted code, machine code) that embodies one or more computer functions described herein, executable instructions, and/or the like may be used to control certain aspects of the operation of the computing entity 200 with the assistance of the processing element 205 and operating system.

As indicated, in some embodiments, the computing entity 200 may also include one or more network interfaces 220 for communicating between various computing entities such as by communicating data, code, content, information, and/or similar terms used herein interchangeably that may be transmitted, received, operated on, processed, displayed, stored, and/or the like. Such communication may be executed using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. In some embodiments, the computing entity 200 communicates with another computing entity for uploading or downloading data or code. Similarly, the computing entity 200 may be configured to communicate via wireless external communication networks using any of a variety of protocols, such as general packet radio service (GPRS), Universal Mobile Telecommunications System (UMTS), Code Division Multiple Access 2000 (CDMA2000), CDMA2000 1X (1xRTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.11 (Wi-Fi), Wi-Fi Direct, 802.16 (WiMAX), ultra-wideband (UWB), infrared (IR) protocols, near field communication (NFC) protocols, Wibree, Bluetooth protocols, wireless universal serial bus (USB) protocols, and/or any other wireless protocol.

Although not shown, the computing entity 200 may include, or be in communication with, one or more input elements, such as a keyboard input, a mouse input, a touch screen/display input, motion input, movement input, audio input, pointing device input, joystick input, keypad input, and/or the like. The computing entity 200 may also include, or be in communication with, one or more output elements (not shown), such as audio output, video output, screen/display output, motion output, movement output, and/or the like.

### Example External Computing Entity

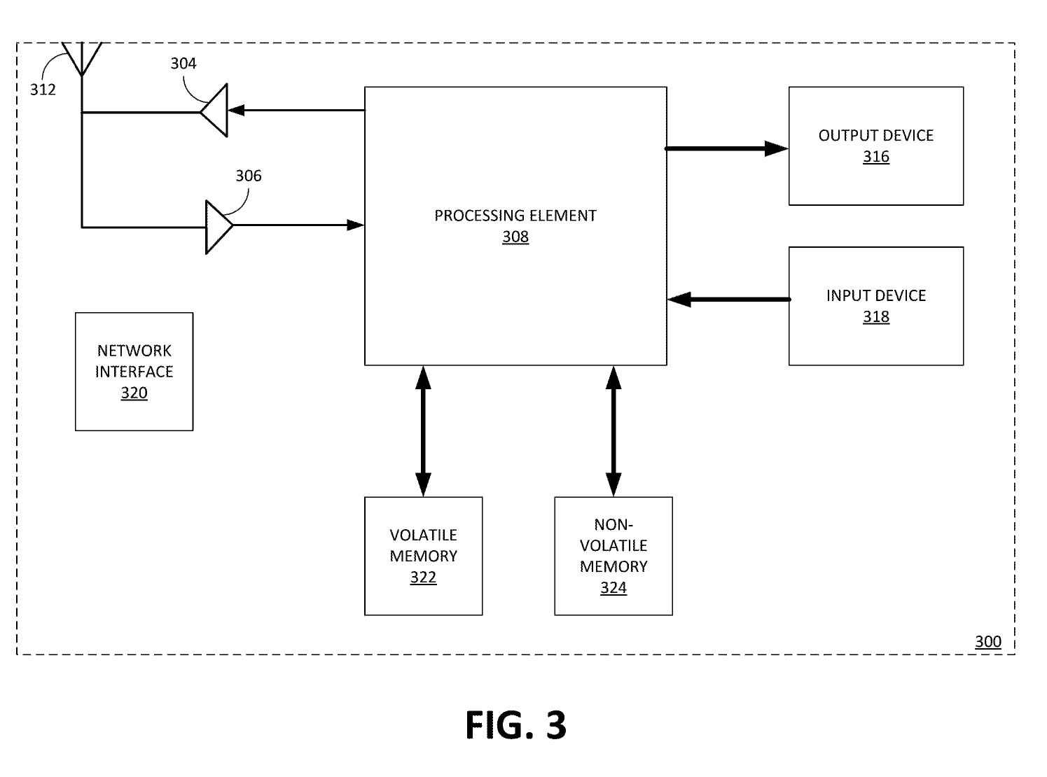

FIG. 3 provides an example computing entity in accordance with some embodiments of the present disclosure. In general, FIG. 3 may be representative of components of any of the modules/systems described herein generally enabling processing within each component and communication between the components. In general, the terms device, system, computing entity, entity, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. As shown in FIG. 3, the computing entity 300 may include an antenna 312, a transmitter 304 (e.g., radio), a receiver 306 (e.g., radio), and a processing element 308 (e.g., CPLDs, microprocessors, multi-core processors, coprocessing entities, ASIPs, microcontrollers, and/or controllers) that provides signals to and receives signals from the transmitter 304 and receiver 306, correspondingly. Generally, computing entity 300 may be representative of a device configured for hosting inference module 120, VAE 130, or alternative model 150 in accordance with at least some embodiments of the present invention.

The signals provided to and received from the transmitter 304 and the receiver 306, correspondingly, may include signaling information/data in accordance with air interface standards of applicable wireless systems. In this regard, the computing entity 300 may be capable of operating with one or more air interface standards, communication protocols, modulation types, and access types. More particularly, the computing entity 300 may operate in accordance with any of a number of wireless communication standards and protocols, such as those described above with regard to the computing entity 200. In some embodiments, the computing entity 300 may operate in accordance with multiple wireless communication standards and protocols, such as UMTS, CDMA2000, 1xRTT, WCDMA, GSM, EDGE, TD-SCDMA, LTE, E-UTRAN, EVDO, HSPA, HSDPA, Wi-Fi, Wi-Fi Direct, WiMAX, UWB, IR, NFC, Bluetooth, USB, and/or the like. Similarly, the computing entity 300 may operate in accordance with multiple wired communication standards and protocols, such as those described above with regard to the computing entity 200 via a network interface 320.

Via these communication standards and protocols, the computing entity 300 may communicate with various other entities using mechanisms such as Unstructured Supplementary Service Data (USSD), Short Message Service (SMS), Multimedia Messaging Service (MMS), Dual-Tone Multi-Frequency Signaling (DTMF), and/or Subscriber Identity Module Dialer (SIM dialer). The computing entity 300 may also download code, changes, add-ons, and updates, for instance, to its firmware, software (e.g., including executable instructions, applications, program modules), and operating system.

The computing entity 300 may also comprise a user interface (that may include an output device 316 (e.g., display, speaker, tactile instrument, etc.) coupled to a processing element 308) and/or a user input interface (coupled to a processing element 308). For example, the user interface may be a user application, browser, user interface, and/or similar words used herein interchangeably executing on and/or accessible via the computing entity 300 to interact with and/or cause display of information/data from the computing entity 200, as described herein. The user input interface may comprise any of a plurality of input devices 318 (or interfaces) allowing the computing entity 300 to receive code and/or data, such as a keypad (hard or soft), a touch display, voice/speech or motion interfaces, or other input device. In some embodiments including a keypad, the keypad may include (or cause display of) the conventional numeric (0-9) and related keys (#, *), and other keys used for operating the computing entity 300 and may include a full set of alphabetic keys or set of keys that may be activated to provide a full set of alphanumeric keys. In addition to providing input, the user input interface may be used, for example, to activate or deactivate certain functions, such as screen savers and/or sleep modes.

The computing entity 300 may also include volatile memory 322 and/or non-volatile memory 324, which may be embedded and/or may be removable. For example, the non-volatile memory 324 may be ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like. The volatile memory 322 may be RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like. The volatile and non-volatile memory may store databases, database instances, database management systems, data, applications, programs, program modules, scripts, code (source code, object code, byte code, compiled code, interpreted code, machine code, etc.) that embodies one or more computer functions described herein, executable instructions, and/or the like to implement the functions of the computing entity 300. As indicated, this may include a user application that is resident on the computing entity 300 or accessible through a browser or other user interface for communicating with the computing entity 200 and/or various other computing entities.

In another embodiment, the computing entity 300 may include one or more components or functionalities that are the same or similar to those of the computing entity 200, as described in greater detail above. As will be recognized, these architectures and descriptions are provided for example purposes only and are not limited to the various embodiments.

In various embodiments, the computing entity 300 may be embodied as an artificial intelligence (AI) computing entity. Accordingly, the computing entity 300 may be configured to provide and/or receive information/data from a user via an input/output mechanism, such as a display, a camera, a speaker, a voice-activated input, and/or the like. In certain embodiments, an AI computing entity may comprise one or more predefined and executable program algorithms stored within an onboard memory storage module, and/or accessible over a network. In various embodiments, the AI computing entity may be configured to retrieve and/or execute one or more of the predefined program algorithms upon the occurrence of a predefined trigger event.

### Example EEG Calibration System

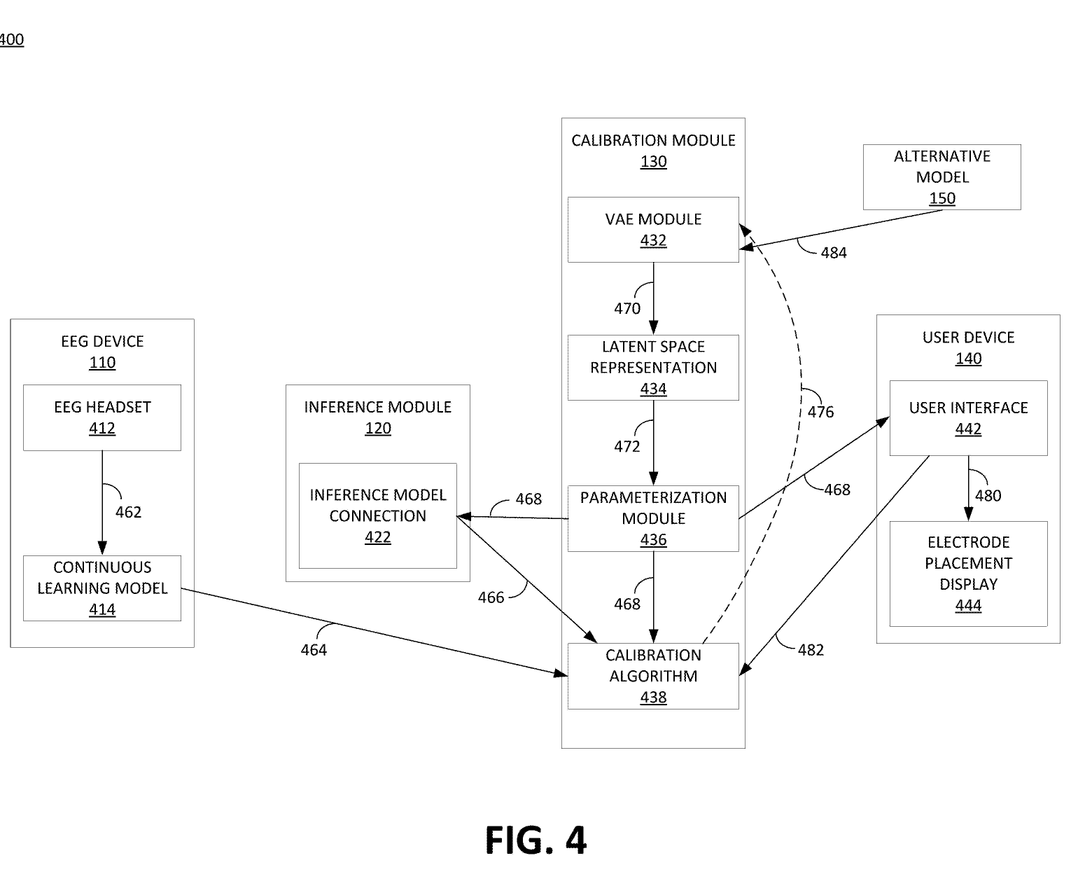

FIG. 4 depicts dataflow within an EEG system 400 in accordance with at least some embodiments of the present invention. As depicted, the EEG system 400 includes EEG device 110, inference module 120, calibration module 430, user device 140, and alternative model 150. FIG. 4 generally depicts one example of how data is transferred between components of the EEG system 400 in accordance with at least one embodiment of the present invention.

As depicted, EEG device 110 includes an EEG headset 412 and a continuous learning model 414. EEG headset 412 may be any EEG device configured to monitor an individual’s brain activity as described with respect to FIG. 1. Continuous learning model 414 may be a machine learning model configured to integrate EEG data 462, as provided by EEG headset 412, into a calibration algorithm, thereby enabling continuous adaptation. In at least some embodiments, continuous learning model 414 may be configured to adjust dynamically to new brain states or conditions. Continuous learning model 414 may include one or more corresponding application programming interfaces configured to allow communication between the EEG headset 412 and the continuous learning model 414. It should be appreciated that, while continuous learning model 414 is depicted as a part of EEG device 110, there exist additional embodiments wherein continuous learning model 414 is instead an integrated part of variational autoencoder 130, inference module 120, or any other component of EEG system 400. Notably, continuous learning model 414 functions as intended as long as it is capable of receiving EEG data 462 for processing. With respect to the depicted embodiment, continuous learning model 414 is configured to provide new data integration 464 to calibration algorithm 438.

As depicted, inference module 120 includes an inference model connection 422. Inference model connection 422 may be an application configured to leverage a connected (or in some embodiments, an integrated) inference model configured to apply optimized EEG parameters to tasks such as emotional inference or motor intent. In at least some embodiments, inference model connection 422 is configured to receive EEG parameters 468 from a parameterization module 436 and process said parameters to provide inference model feedback 466 to calibration algorithm 438. In at least some embodiments, inference module 120 may be configured to additionally process received supervisory labels. In general, inference model connection 422 is configured to apply optimized EEG parameters, such as preferred placements, to various EEG tasks such as emotional inference or motor intent. Preferred parameters such as preferred placements may vary according to task and as such, inference model connection 422 may be configured to manage appropriate parameterization relative to a current/selected/preferred task.

As depicted, calibration module 430 includes a VAE module 432, a latent space representation 434, a parameterization module 436, and a calibration algorithm 438. In at least some embodiments, calibration module 430 is configured for various communications with alternative model 150, user device 140, inference module 120, and EEG device 110. In general, calibration module 430, via its various components, is configured to process varied EEG-related data to ultimately provide an optimized EEG calibration.

VAE module 432 is configured to receive one or more control parameters 476 associated with a current iteration of calibration algorithm 438. In at least some embodiments, VAE module 432 is additionally configured to receive EEG data, either directly from EEG headset 412 or via one of the intermediate modules configured to communicated with VAE module 432 directly. VAE module 432 may be configured to process EEG data using either a standard VAE or a fully spiking VAE. In at least some embodiments, VAE module 432 is configured to construct and retain latent space representation 434. Latent space representation 434 may be configured as a compressed, lower dimensional representation of data received and/or generated by the VAE module 432, such as the EEG data and corresponding set of control parameters. In at least some embodiments, the VAE module is configured to compress EEG data into latent space representation 434 and analyze the latent space representation 434 to determine which electrode placements are most useful for predicting one or more desired outcomes. The EEG data processed by the VAE module 432 may include raw EEG data/signals, electrode placement information provided by either the EEG device 110 itself or by manual user input, and a third variable indicative of a current system goal (such as, for example, a psychological state the system is attempting to infer). In some embodiments, such a third variable may be a discrete variable, such as a variable indicating whether the individual is surprised or unsurprised. In some embodiments, the third variable may be a continuous variable, such as a scale measuring the individual’s ability to maintain a “clear head” or a calm state. VAE module 432 may additionally be configured to sample latent space representation 434 to identify preferred control parameters. In at least some embodiments, VAE module 432 is configured to generate latent space data 470 describing the latent space representation 434. Latent space data 470 may additionally be provided to parameterization module 436.

Parameterization module 436 is configured to process latent space representation 434 and/or latent space data 470 to generate EEG parameters 468. In at least some embodiments, parameterization module 436 is configured to provide the EEG parameters 468 to inference module 120 (via inference model connection 422, for example). In at least some embodiments, parameterization module 436 is configured to provide EEG parameters 468 to calibration algorithm 438. Parameterization module 436 may be configured to transform or otherwise provide EEG parameters 468 in a format usable by either of inference module 120 or calibration algorithm 438. In at least some embodiments, parameterization module 436 is configured to provide EEG parameters 468 responsive to a request received from either of inference module 120 or calibration algorithm 438. Parameterization module 436 may be configured to automatically initiate transmission of the EEG parameters 468 whenever the parameters are updated based on a change to the latent space representation 434.

Calibration algorithm 438 may be configured to refine the EEG parameters 468 and/or the latent space representation 434. In at least some embodiments, calibration algorithm 438 is configured to apply one or more uncertainty quantification techniques. Uncertainty quantification (UQ) is a systematic approach to identifying and managing sources of error and variability within a system. Calibration algorithm 438 may use UQ techniques to identify and account for inherent uncertainties in both the data and the models utilized by the system. In at least some embodiments, calibration algorithm 438 is configured to identify regions within the latent space representation 434 which require more detailed exploration (such as spaces which have not been sampled considerably/exhibit insufficient sampling, or regions which have yielded considerable successes with respect to a desired determination, for example). Identifying regions in such a fashion enables refined electrode placement strategies by focusing on areas in the latent space that are most informative for a target psychological state, for example, while also accounting for inherent variability in EEG data. Calibration algorithm 438 may be configured to provide refined control parameters 476 to VAE module 432.

In at least some embodiments, calibration algorithm 438 is configured to employ manifold discovery techniques. Manifold discovery augments an algorithm’s ability to identify key patterns in complex data by tracing out the most important structures within the data (akin to identifying critical regions on a map). Once these important regions/structures are identified, calibration algorithm 438 may be configured to utilize targeted sampling techniques within these regions to gather additional data, providing increased granularity in the most effective regions and consequently improving performance.

In at least some embodiments, calibration algorithm 438 is configured to leverage Bayesian methods for quantifying uncertainty and guiding electrode placement. Bayesian methods are probabilistic techniques configured to guide a system’s decision making in the presence of uncertainty. In at least some embodiments, calibration algorithm 438 is configured to utilize Bayesian methods which minimize focus on less relevant states, maximize the probability of accurately distinguishing between psychological states, and/or continuously update predictions based on incoming data. With respect to the described implementations, utilizing Bayesian methods ensures the system optimizes according to the most meaningful signals available.

In at least some embodiments, such as those where a limited number of electrodes (e.g., less than five) are available, calibration algorithm 438 may be configured to generate optimized electrode placements based on the limited electrode availability. For example, when determining optimal placements for the defined number of electrodes, the optimal placement of a selected electrode may be specific to that defined number of electrodes; in other words, an “optimal placement” determined with respect to one or more two electrodes may not necessarily also be an “optimal placement” determined with respect to one or more three electrodes. In some embodiments, the one or more machine learning models may be configured to prioritize or otherwise process certain parameters differently with respect to a limited set of available electrodes; for example, if one or more electrodes is too small to ensure optimal placement with respect to every region of the brain, the one or more machine learning models may be configured to generate placements which ensure a baseline level of operation and/or which ensure coverage of a selected region of interest, for example.

As depicted, user device 140 includes a user interface 442 and an electrode placement display 444. In at least some embodiments, user interface 442 and electrode placement display 444 are integrated together in a graphical user interface configured to visualize electrode placement and performance in real-time, while also enabling user interactions with the system. It should be appreciated that any of the functions of the user interface 442 or the electrode placement display 444 individually may be attributed to a combined GUI instead in certain embodiments. In at least some embodiments, user interface 442 is configured to guide users through a calibration process corresponding to the EEG device 110 and facilitate intuitive optimization of electrode placement.

In at least some embodiments, user interface 442 is configured to receive EEG parameters 468 from parameterization module 436. User interface 442 may be configured to process the received EEG parameters 468 to generate a display indicating preferred areas for electrode placement on an individual. In at least some embodiments, user interface 442 is configured to provide one or more rankings of the indicated preferred areas for electrode placements based on a desired inference task. User interface 442 may be configured to receive user interactions and/or selections with respect to electrode placements, desired inference task(s), and the like, and provide real-time feedback on electrode performance responsive to the user interactions. For example, when a user selects or changes a desired inference task selection, the user interface 442 may be configured to provide real-time feedback indicating how current electrode positions and/or potential electrode positions perform with respect to the newly selected inference task. This dynamic guidance enables a user to maximize the system’s performance by strategically positioning the available electrodes based on their importance to the inference task. In some embodiments, user interface 442 is configured to provide electrode placement recommendations based on the desired inference task and the available electrodes. For example, user interface 442 may display an image depicting preferred locations for the available electrodes, thereby enabling the user to position the electrodes according to the displayed image. Such an image may be displayed via electrode placement display 444.

In at least some embodiments, user interface 442 is configured to generate a color-coded scalp map corresponding to the preferred electrode placement(s). In at least some embodiments, the color-coded scalp map indicates optimized placements based on signal quality. User interface may be configured to display one or more metrics for each electrode position and/or for the overall system based on all of the electrode positions. In at least some embodiments, the one or more display metrics include predicted accuracy, uncertainty estimates, and/or signal contribution(s) corresponding to each electrode position. User interface 442 may be configured to enable a user to adjust a depicted electrode position. In such embodiments, user interface 442 may additionally be configured to display an indication of how the user’s updates to electrode position(s) affect model performance/model accuracy.

In at least some embodiments, user interface 442 is configured to enable a user to input custom psychological state labels, prompting the system to optimize electrode placement for maximizing differentiation between these states. In such embodiments, user interface 442 may visualize how these inputs alter the system’s recommendations, thereby enabling ease of exploring specific hypotheses or clinical applications. Such visualization facilitates rapid experimentation with various state classifications, thereby enhancing the system’s versatility for specialized research needs.

In at least some embodiments, such as those where no specific target is defined, user interface 442 may work in conjunction with inference module 120, for example, to evaluate calibration module 430’s output to determine an inferred target and refine electrode placement further. User interface 442 may be configured to provide a generalized optimization in the absence of a specific target, such that the recommended placements provide a best overall performance relative to a plurality of targets.

In at least some embodiments, user interface 442 may be configured to provide display data 480 to electrode placement display 444. Such display data 480 may include any information as described above as being displayed to a user. User interface 442 may be configured to provide user feedback information 482 to calibration algorithm 438, such that the calibration algorithm can calibrate the electrode placement optimizations according to any received user feedback.

Alternative model 150 may be an algorithm or model differing from calibration algorithm 438 configured to provide a secondary set of refined parameters 484. In certain embodiments, such as those where model performance is unknown or not yet validated, application of an alternative model such as alternative model 150 provides a flexible setup. In at least some embodiments, such as embodiments wherein the VAE module 432 is configured to perform both calibration and inference, alternative model 150 may be leveraged by the VAE module 432 to utilize supervisory labels for dual calibration and inference. It should be appreciated that there are many embodiments in which the use of an alternative model 150 is not necessary.

In at least some embodiments, components of the EEG system 400 are implemented on neuromorphic hardware components such as a neuromorphic chip. Neuromorphic hardware is designed to mimic the structure and function of biological neural networks. Unlike traditional von Neumann architectures, neuromorphic chips are optimized for parallel processing and energy efficiency, making them well-suited for neural network computations and brain-inspired algorithms. In at least some embodiments, the neuromorphic hardware components enable parallel processing capabilities, low power consumption, event-driven computation, and co-location of memory ant processing. Neuromorphic hardware components may additionally provide efficient processing of neural network algorithms, real-time processing of complex neural signals, and efficient handling of sparse, event-driven data similar to biological neural systems.

In at least some embodiments, VAE module 432 is configured as a fully spiking variational autoencoder (FSVAE). In such embodiments, VAE module 432 uses spiking neurons that activate only when they reach a certain threshold, thereby mimicking biological neurons. In such embodiments, VAE module 432 processes information in discrete events rather than continuous values, aligning with the event-driven nature of neuromorphic hardware. In such embodiments, VAE module 432 can naturally incorporate temporal dynamics, thereby enabling increased performance when processing time-series data such as EEG signals.

In embodiments wherein the VAE module 432 is configured as a fully spiking variational autoencoder, the EEG system can perform deep learning tasks in real-time without relying on external hardware, thereby providing a more self-sufficient EEG device system independent from larger computational frameworks. Such embodiments additionally provide improved efficiency and power consumption when processing EEG data, electrode placements, and psychological state variables. Such embodiments may additionally enable real-time feedback to user adjustments, thereby improving the accuracy and timeliness of electrode placement suggestions. Such embodiments may additionally enable longer operation times without frequent recharging in embodiments where the EEG device operates on battery power, thereby increasing portability and usability. Such embodiments may further enable increased scalability compared to conventional EEG systems/devices.

### Examples of Certain Terms

In some embodiments, the terms “user system,” “user device,” “user computing device,” “user computing entity,” and the like may refer to any system configured to run an EEG calibration method as described herein. In general, the user system may refer to any system capable of providing an interface to a user device via which a user can interact with an EEG optimization application and/or a display of optimized EEG placements. User systems as described herein enable a user to view electrode performance information, view data received from an EEG device, select desired inference tasks, and/or interact with one or more selectable features corresponding to an EEG system.

In some embodiments, the term “calibration module” refers to any application, device, or system configured to refine a latent space representation of electrode placement optimization data. In at least some embodiments, a calibration module is configured to run a calibration algorithm for refining the described latent space. The calibration module may be configured to apply uncertainty quantification techniques for refining the described latent space and further explore the latent manifold. The calibration module may be configured to utilize manifold discovery techniques, manifold sampling techniques, manifold investigation techniques, Bayesian algorithms, and/or any other uncertainty quantification techniques known in the art.

In some embodiments, the terms “inference module,” “task inference module,” and the like may refer to any application, system, or device configured to use optimized parameters to predict a patient’s brain state or features. An inference module may be configured to provide feedback to a VAE module corresponding to predicted psychological/brain-state features. In general, an inference module may be configured to execute an optimized inference model to predict a patient/subject’s state.

In some embodiments, the term “VAE module” may refer to any application, system, or device configured to leverage a variational autoencoder for optimizing electrode placement. A VAE module as used herein may correspond to any entity configured to process or compress EEG data into a latent space representation. VAE modules as described herein may be configured to process EEG signals, electrode placements, and a system goal variable to provide a latent space representation for refinement with respect to optimized electrode placement parameters.

### Overview, Technical Improvements, and Technical Advantages

Various embodiments of the present disclosure make important technical contributions to fields and/or environments leverage brain-computer interface (BCI) applications.

For example, various embodiments of the present disclosure improve systems which monitor brain activity using an EEG device configured with one or more electrodes placed on an individual. EEG devices range from research-grade systems with many electrodes to compact commercial headsets; as such, optimizing electrode placement is crucial for EEG systems inferring brain states, particularly for commercial headsets with fewer electrodes. Effective electrode placement as enabled by the embodiments of the present disclosure improves setup ease, comfort, affordability, and performance of EEG devices.

Various embodiments of the present invention provide improvements to passive BCI applications, wherein the system observes and interprets natural brain states without requiring user learning or control. Passive BCI applications avoid complexities associated with active BCIs, such as frequent calibration and modeling of user adaptation. Embodiments of the system(s) described herein include an algorithm and a graphical user interface (GUI) to assist users in optimizing electrode placement, ensuring good performance regardless of hardware availability. Such optimization is particularly relevant for EEG systems which can employ as few as 8 or 16 electrodes. In at least some embodiments, a variational autoencoder (VAE) is utilized for electrode optimization, incorporation of uncertainty qualification techniques, design of the user interface, and implementation on neuromorphic hardware. Embodiments of the present invention improve the accessibility and effectiveness of EEG technology for a range of passive BCI applications.

### Example System Operations

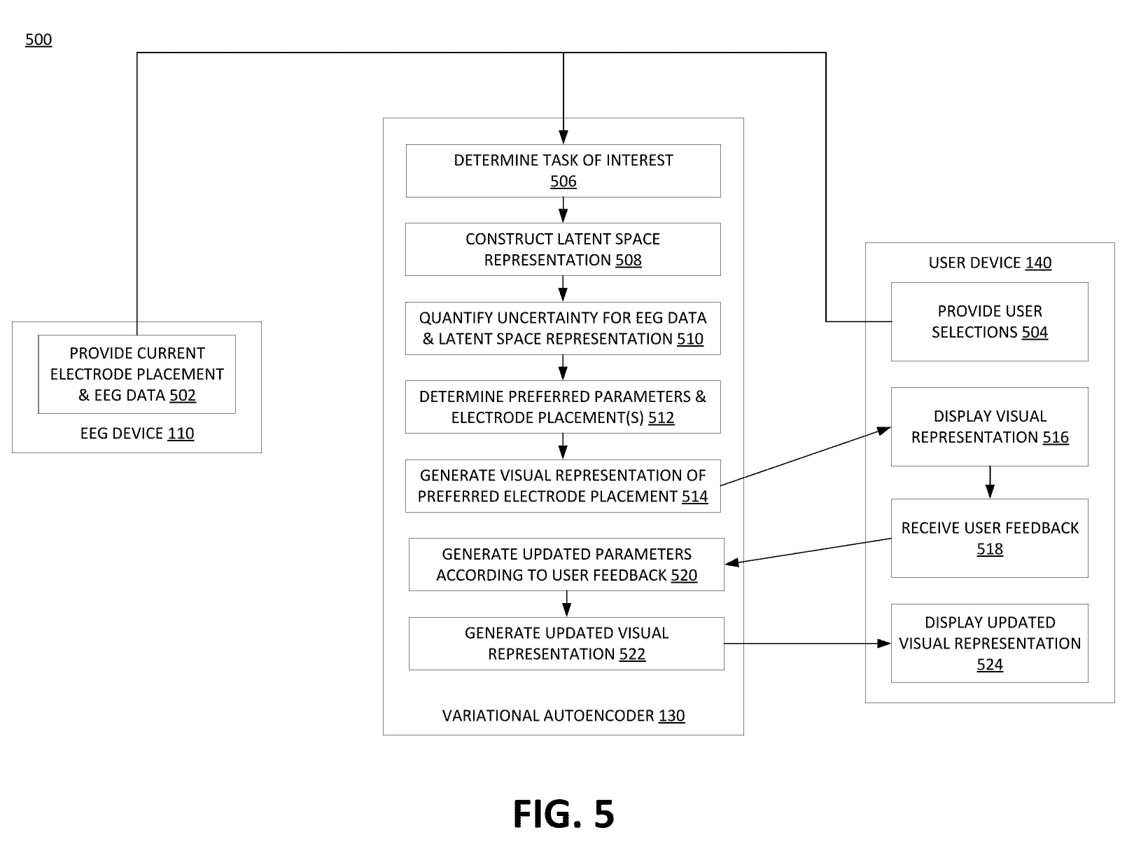

FIG. 5 depicts an example process 500 executed across a plurality of components including an EEG device 110, a VAE module 432, and a user device 140 in accordance with at least some embodiments of the present invention. As depicted, process 500 includes providing (502) current electrode placement data and EEG data, providing (504) one or more user selections, determining (506) a task of interest, generating (508) a latent space representation, determining (510) uncertainty for the EEG data and the latent space representation, generating (512) preferred parameters and electrode placement(s), generating (514) a visual representation of the preferred electrode placement, displaying (516) the visual representation, receiving (518) user feedback, generating (520) updated parameters according to the user feedback, generating (522) an updated visual representation of the preferred electrode placement, and displaying (524) the updated visual representation. In at least some embodiments, one or more of the components may be implemented on neuromorphic hardware (such as a neuromorphic chip, for example).

At step 502, EEG device 110 is configured to provide current electrode placement data and EEG data. In at least some embodiments, EEG device 110 provides EEG data to any of VAE module 432 and user device 140 via one or more network transmissions. In at least some embodiments, EEG device 110 is configured to provide real time EEG data via ongoing transmission(s). In other embodiments, EEG device 110 may be configured to provide EEG data at predetermined intervals. EEG device 110 may be configured to provide EEG data responsive to a query received from VAE module 432 and/or user device 140. In at least some embodiments, EEG device 110 is additionally configured to provide electrode placement data. Electrode placement data may include an indication of a number of electrodes available for use by EEG device 110, current placement information corresponding to one or more electrodes of EEG device 110, and/or current performance data corresponding to the current electrode placement.

At step 504, user device 140 is configured to provide one or more user selections. The one or more user selections may be provided via an interface, such as a GUI, of user device 140, and may correspond to a selection of a current task for analysis. In at least some embodiments, the one or more user selections include one or more user indicated placement preferences. For example, the one or more user indicated placement preferences may include an indication of one or more areas in which an electrode should not be placed (due to an injury, for example). The one or more user selections may additionally correspond to a ranking or weighting of one or more tasks for analysis, such that the system can provide preferred electrode placement locations based on the relative importance of the one or more tasks for analysis as indicated by the weighting.

At step 506, VAE module 432 is configured to determine a task of interest. In at least some embodiments, such as those wherein the user has provided one or more tasks of interest, VAE module 432 makes said determination based on the user selections. In at least some embodiments, such as those wherein the user has provided a task of interest, VAE module 432 may be configured to provide recommended related task(s) for the user’s consideration. In such embodiments, VAE module 432 may be configured to provide such a recommendation to user device 140 for display, wherein user device 140 subsequently enables the user to confirm, deny, or alter such a recommendation. In at least some embodiments, such as those where no task of interest has been selected by a user, VAE module 432 is configured to infer a task. In at least some embodiments, VAE module 432 may process the EEG data to identify a current state of the user and a corresponding task. For example, VAE module 432 may process the EEG data and determine that the data is consistent with a user exhibiting altered motor function, and as such, VAE module 432 may determine that the current state of the user indicates a preferred task of interest corresponds to motor intent.

At step 508, VAE module 432 is configured to generate a latent space representation. In at least some embodiments, VAE module 432 is configured to construct and retain a latent space representation configured as a compressed, lower dimensional representation of data received and/or generated by the VAE module 432, such as the EEG data and a corresponding set of control parameters. In at least some embodiments, the VAE module is configured to compress EEG data into the latent space representation and analyze the latent space representation to determine which electrode placements are most useful for predicting one or more desired outcomes.

At step 510, VAE module 432 is configured to determine uncertainty for the EEG data and the latent space representation. In at least some embodiments, VAE module 432 is configured to leverage one or more uncertainty quantification (UQ) techniques, including (but not limited to) manifold discovery, manifold sampling, manifold investigation, and/or Bayesian algorithms. In general, VAE module 432 may be configured to determine a level of uncertainty based on the EEG data and the corresponding latent space representation. The level of uncertainty may be determined according to EEG signal strength, EEG data quality/consistency, latent space sampling, and the like.

Uncertainty quantification techniques may be configured to measure and/or predict sensor-level uncertainty, such as electrode-scalp contact impedance, motion-induced artifacts, thermal and electronic noise, and/or electrode polarization effects. In some embodiments, the uncertainty quantification techniques include noise modeling (using additive Gaussian/colored noise models and/or empirical noise covariance estimated from baseline segments), impedance-aware weighting (by assigning uncertainty weights to channels based on measured impedance and visualizing impedance as confidence bars or transparency on electrode markers), bootstrap resampling (by using repeated resampling of time windows to estimate variance of metrics such as power spectral density, ERP amplitudes, connectivity metrics, and the like).

In some embodiments, uncertainty quantification techniques may be configured to measure and/or predict spatial uncertainty in electrode positioning, based on variables such as cap misalignment, head shape variability, manual placement error, and/or elastic deformation of caps. Such techniques may be configured using landmark perturbation models configured to tread fiducials (nasion, inion, preauricular points) as random variables and propagate error through coordinate registration. In some embodiments, Monte Carlo electrode jitter is utilized to randomly perturb electrode positions within realistic bounds (e.g., 3-7mm), and recompute downstream metrics for each electrode.

In some embodiments, uncertainty quantification techniques may be configured to model lead-field uncertainty based on variables such as tissue conductivity values, skull thickness and anisotropy, and segmentation errors in MRI-derived models. Appropriate techniques for modeling lead-field uncertainty may include, but are not limited to, Stochastic forward modeling (by sampling conductivity parameters from distributions and generating ensembles of lead-field matrices), sensitivity analysis (by determining partial derivatives of scalp potentials with respect to tissue parameters and identifying which uncertainties dominate placement sensitivities), and/or model averaging (by averaging predictions over multiple head models and weighting by anatomical similarity or likelihood).

At step 512, VAE module 432 is configured to generate preferred parameters and electrode placement(s). In at least some embodiments, VAE module 432 is configured to sample the latent space representation of the EEG data to identify preferred parameters. The latent space representation of the EEG data may include a compressed representation of data provided by each electrode of the EEG device 110. As such, the latent space representation may include sections corresponding to each electrode (and subsequently, each electrode location) such that portions of the EEG data are identifiable based on the providing electrode. In at least some embodiments, while the data may not be labeled as having been “provided” by each electrode necessarily, VAE module 432 may be able to infer which electrode has most heavily influenced a portion of the data. For example, when analyzing motor intent data, VAE module 432 can infer that an electrode whose current placement is closest to a known optimal placement location for motor intent determination likely provides the most informative motor intent data. In at least some embodiments, VAE module 432 is configured determine a signal strength associated with each electrode in a current electrode position. If VAE module 432 determines that an electrode is exhibiting poor signal strength because of its positioning, the determined optimization parameters and electrode placement(s) may include an instruction to reposition said electrode to a more effective position for the determined task. For example, if VAE module 432 determines that electrodes positioned proximate to a first region of an individual’s head generally provide more reliable data when analyzing motor intent, VAE module 432 may be configured to recommend repositioning of an underperforming electrode to a position closer to said first region of the individual’s head. In at least some embodiments, such as those where a required distance must be maintained between the electrodes, VAE module 432 is configured to recommend electrode placements that optimize electrode proximity to a preferred region while also maintaining the required distance from one another. For example, VAE module 432 may be configured to minimize aggregate distance from a preferred region across the electrodes while retaining the require space in between each pair of electrodes.

At step 514, VAE module 432 is configured to generate a visual representation of the preferred electrode placement. In at least some embodiments, VAE module 432 is configured to generate a rendering of an individual’s head including one or more preferred electrode placement locations. In at least some embodiments, such as those where the EEG device or other system component includes a camera or other media capture device, VAE module 432 may be configured to generate a rendering of the preferred electrode placement locations with respect to an image or video frame of the individual’s head. In at least some embodiments, VAE module 432 is configured to generate a rendering of the current electrode placement along with a rendering of the preferred electrode placement such that the user may visibly decipher how far to move each electrode, for example. In general, VAE module 432 is configured to generate an image or other representation of the determined preferred electrode placement locations.

At step 516, user device 140 is configured to display the visual representation. User device 140 may be configured to receive the visual representation of the preferred electrode placement from the VAE module 432. In at least some embodiments, user device 140 is configured to display the rendering of the individual’s head including one or more preferred electrode placement locations. In at least some embodiments, such as those where the EEG device or other system component includes a camera or other media capture device, user device 140 may be configured to display the rendering of the preferred electrode placement locations with respect to an image or video frame of the individual’s head. In at least some embodiments, user device 140 is configured to display the rendering of the current electrode placement along with a rendering of the preferred electrode placement such that the user may visibly decipher how far to move each electrode, for example. In general, user device 140 is configured to display an image or other representation of the determined preferred electrode placement locations as received from VAE module 432.

At step 518, user device 140 is configured to receive user feedback corresponding to the displayed visual representation. In at least some embodiments, user device 140 is configured to receive user interactions with the displayed preferred electrode placement locations, such that the user may alter or otherwise relocate the placements directly via the rendering. In at least some embodiments, user device 140 is configured to provide one or more selectable options to the user, such that the user may indicate, via the selectable options, an updated optimization. For example, user device 140 may be configured to provide alternative task options to the user such that the user may select a different task with respect to which the electrode placement locations will be preferred. In at least some embodiments, user device 140 is configured to provide one or more sliders via which a user can indicate preferences on a spectrum. For example, in an embodiment where VAE module 432 is capable of providing placement optimizations with increased confidence with slightly increased processing time, the user may be provided a slider via which they can indicate a preferred tradeoff between confidence and speed, thereby informing the actions of the VAE module 432 accordingly. In at least some embodiments, user device 140 may be configured to provide one or more checkboxes to the user via which the user can select (or deselect) one or more corresponding tasks for optimization. When selected, a corresponding task will inform/impact the optimization of the electrode placements; similarly, when deselected/not selected, a corresponding task will not be considered and therefore will not inform/impact the optimization of the electrode placements.

At step 520, VAE module 432 is configured to generate updated parameters according to the user feedback. In at least some embodiments, VAE module 432 is configured to update the one or more preferred electrode placement locations based at least in part on the user feedback. In an embodiment where the user feedback corresponds to adjustments to the previously provided preferred electrode placement locations, generating updated parameters according to the user feedback may include updating said previously provided preferred electrode placement locations according to the user feedback rather than generating a new set of preferred electrode placement locations. In an embodiment where the user feedback indicates larger scale updates to the parameters, such as adjusting the optimization to reflect a different task of interest, generating updated parameters may include generating an entirely new set of preferred electrode placement locations.

At step 522, VAE module 432 is configured to generate an updated visual representation of the preferred electrode placement. In at least some embodiments, VAE module 432 is configured to generate a rendering of an individual’s head including one or more updated preferred electrode placement locations. In at least some embodiments, such as those where the EEG device or other system component includes a camera or other media capture device, VAE module 432 may be configured to generate a rendering of the updated preferred electrode placement locations with respect to an image or video frame of the individual’s head. In at least some embodiments, VAE module 432 is configured to generate a rendering of the current electrode placement along with a rendering of the updated preferred electrode placement such that the user may visibly decipher how far to move each electrode, for example. In general, VAE module 432 is configured to generate an image or other representation of the updated preferred electrode placement locations.

At step 524, user device 140 is configured to displaying (524) the updated visual representation. User device 140 may be configured to receive the visual representation of the updated preferred electrode placement from the VAE module 432. In at least some embodiments, user device 140 is configured to display the rendering of the individual’s head including one or more updated preferred electrode placement locations. In at least some embodiments, such as those where the EEG device or other system component includes a camera or other media capture device, user device 140 may be configured to display the rendering of the updated preferred electrode placement locations with respect to an image or video frame of the individual’s head. In at least some embodiments, user device 140 is configured to display the rendering of the current electrode placement along with a rendering of the updated preferred electrode placement such that the user may visibly decipher how far to move each electrode, for example. In general, user device 140 is configured to display an image or other representation of the updated preferred electrode placement locations as received from VAE module 432.

In at least some embodiments, the process 500 further includes causing performance of one or more prediction-based actions. The one or more prediction-based actions may be triggered responsive to completion of any number of the steps described with respect to process 500; for example, responsive to receiving (518) user feedback corresponding to the displayed visual representation, in addition to generating (520) updated parameters according to the user feedback, VAE module 432 may further be configured to generate a placement profile associated with any of (i) a corresponding user or individual, (ii) a corresponding number of available electrodes, (iii) a selected region of interest, and so on.

As another example, responsive to determining (510) uncertainty for the EEG data and the latent space representation, the VAE module 432 may be configured to trigger one or more notifications to an authorized user indicating the determined uncertainty. In some embodiments, the VAE module 432 may be configured to trigger such notification(s) responsive to determining that the uncertainty for the EEG data and the latent space representation exceeds an allowable threshold. The one or more notifications may further include one or more recommended actions associated with the determined uncertainty. For example, in an embodiment wherein the EEG data is limited (falls below a preferred threshold), the one or more notifications may further include a recommendation to obtain additionally available EEG data, either from a same device or one or more additionally connected devices. In some embodiments, responsive to detecting limited EEG data, the VAE module 432 may be configured to automatically query, retrieve, or otherwise obtain available additional EEG data from the EEG device 110 and/or user device 140.

As described with respect to process 500, generating a visual representation may comprise utilizing template-based visuals incorporating a standardized scalp coordinate system to create an electrode mapping. Such techniques may begin by generating a 2D scalp map or a 3D head mesh. Once the target head space has been mapped, electrode locations are plotted at the determined optimal “coordinates.” In some embodiments, preferred electrodes may be highlighted using color coding, size emphasis, or labels.

In some embodiments, generating a visual representation may comprise generating an optimization-based heatmap by computing a spatial score for each scalp location based on metrics such as, but not limited to, signal-to-noise ratio, classification accuracy, mutual information, source localization error, and the like. The spatial score may be interpolated across the scalp surface and rendered as a color heatmap on a 2D scalp map or a 3D head mesh to represent the preferred electrode positions.

In some embodiments, generating a visual representation may comprise visualizing preferred electrode clusters as centroids, confidence ellipses, or density plots. The electrode clusters may be presented as bubble plots, probabilistic scalp maps, “top-N channel” overlays, and the like, enabling selection of optimized electrode locations from the preferred electrode clusters, particularly in embodiments where a subject head or scalp has regions which may need to be avoided (e.g., due to injury, etc.).

In some embodiments, such as those where augmented reality and/or procedural guidance technologies are available, generating a visual representation may include registering head landmarks, overlaying electrode targets in real time, and displaying the electrode positions real time through a user device such as a tablet, headset, or camera feed. The displayed visual elements may include floating target dots, distance/error indicators, and/or step-by-step alignment guides.

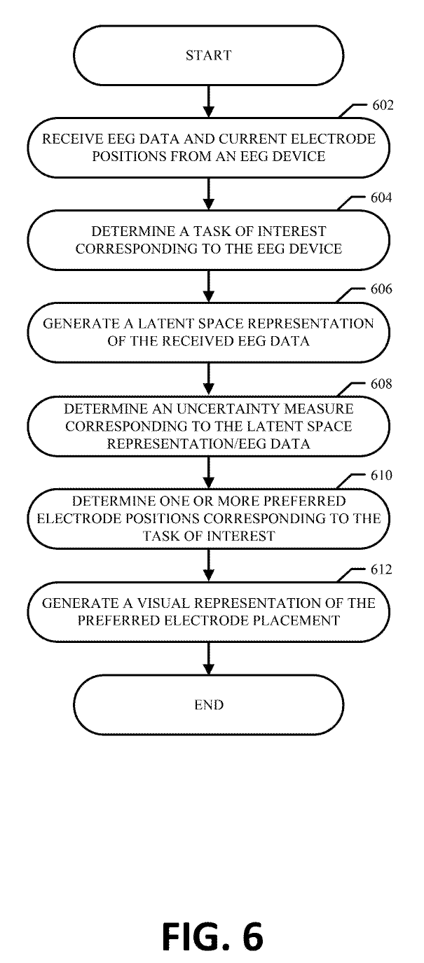

FIG. 6 is a flowchart diagram of an example process 600 for electrode placement optimization in accordance with at least some embodiments of the present invention. As depicted, process 600 includes receiving (602) EEG data and current electrode positions, identifying (604) a task of interest, generating (606) a latent space representation, determining (608) an uncertainty quantification for the latent space representation, determining (610) preferred electrode placements for the task of interest, and generating (612) a visual representation of the preferred electrode placement. Process 600 may enable improved electrode placement with respect to passive BCI applications.

At step 602, VAE module 432 is configured to receive EEG data and one or more current electrode placements. In at least some embodiments, VAE module 432 receives raw EEG data from an EEG device (such as EEG device 110) for processing. In at least some embodiments, VAE module 432 is configured to receive EEG data via one or more network transmissions. Similarly, VAE module 432 may be configured to receive electrode placement data from an EEG device (such as EEG device 110). In at least some embodiments, such as those wherein an EEG device arrangement includes a camera or other media capture device, VAE module 432 receives electrode placement data in the form of an image depicting current electrode placement with respect to an individual. VAE module 432 may utilize one or more image processing techniques to determine current electrode placement locations as depicted with respect to the image.

At step 604, VAE module 432 is configured to identify a task of interest. In at least some embodiments, VAE module 432 is configured to identify one or more BCI tasks associated with a current subject. In at least some embodiments, such as those wherein the user has provided one or more tasks of interest, VAE module 432 identifies the task of interest based on the user selections. In at least some embodiments, such as those wherein the user has provided a task of interest, VAE module 432 may be configured to provide additional recommended related tasks for the user’s consideration. In at least some embodiments, such as those where no task of interest has been selected by a user, VAE module 432 is configured to infer a task. In at least some embodiments, VAE module 432 may process the EEG data to identify a current state of the user and a corresponding task. For example, VAE module 432 may process the EEG data and determine that the data is consistent with a user exhibiting altered motor function, and as such, VAE module 432 may determine that the current state of the user indicates a preferred task of interest corresponds to motor intent.

At step 606, VAE module 432 is configured to generate a latent space representation corresponding to the received EEG data and the one or more current electrode placements. In at least some embodiments, VAE module 432 is configured to construct and retain a latent space representation configured as a compressed, lower dimensional representation of data received and/or generated by the VAE module 432, such as the EEG data and a corresponding set of control parameters. In at least some embodiments, the VAE module 432 is configured to compress EEG data into the latent space representation and analyze the latent space representation to determine which electrode placements are most useful for predicting one or more desired outcomes.

At step 608, VAE module 432 is configured to determine an uncertainty measure for the latent space representation. In at least some embodiments, VAE module 432 is configured to leverage one or more uncertainty quantification (UQ) techniques, including (but not limited to) manifold discovery, manifold sampling, manifold investigation, and/or Bayesian algorithms. In general, VAE module 432 may be configured to determine a level of uncertainty based on the EEG data and the corresponding latent space representation. The level of uncertainty may be determined according to EEG signal strength, EEG data quality/consistency, latent space sampling, and the like.

At step 610, VAE module 432 is configured to determine preferred electrode placements for the task of interest. In at least some embodiments, VAE module 432 is configured to sample the latent space representation of the EEG data to identify preferred electrode placements. The latent space representation of the EEG data may include a compressed representation of data provided by each electrode of the EEG device 110. As such, the latent space representation may include sections corresponding to each electrode (and subsequently, each electrode location) such that portions of the EEG data are identifiable based on the providing electrode. In at least some embodiments, while the data may not be labeled as having been “provided” by each electrode necessarily, VAE module 432 may be able to infer which electrode has most heavily influenced a portion of the data. For example, when analyzing motor intent data, VAE module 432 can infer that an electrode whose current placement is closest to a known optimal placement location for motor intent determination likely provides the most informative motor intent data. In at least some embodiments, VAE module 432 is configured determine a signal strength associated with each electrode in a current electrode position. If VAE module 432 determines that an electrode is exhibiting poor signal strength because of its positioning, the determined optimization parameters and electrode placement(s) may include an instruction to reposition said electrode to a more effective position for the determined task. For example, if VAE module 432 determines that electrodes positioned proximate to a first region of an individual’s head generally provide more reliable data when analyzing motor intent, VAE module 432 may be configured to recommend repositioning of an underperforming electrode to a position closer to said first region of the individual’s head. In at least some embodiments, such as those where a required distance must be maintained between the electrodes, VAE module 432 is configured to recommend electrode placements that optimize electrode proximity to a preferred region while also maintaining the required distance from one another. For example, VAE module 432 may be configured to minimize aggregate distance from a preferred region across the electrodes while retaining the require space in between each pair of electrodes.

At step 612, VAE module 432 is configured to generate a visual representation of the preferred electrode placement. In at least some embodiments, VAE module 432 is configured to generate a rendering of an individual’s head including one or more preferred electrode placement locations. In at least some embodiments, such as those where the EEG device or other system component includes a camera or other media capture device, VAE module 432 may be configured to generate a rendering of the preferred electrode placement locations with respect to an image or video frame of the individual’s head. In at least some embodiments, VAE module 432 is configured to generate a rendering of the current electrode placement along with a rendering of the preferred electrode placement such that the user may visibly decipher how far to move each electrode, for example. In general, VAE module 432 is configured to generate an image or other representation of the determined preferred electrode placement locations.

### Specific Applications

It should be appreciated that, though many of the embodiments as described herein have largely been directed towards electrode placement optimization with respect to passive BCI applications and EEG devices, additional embodiments exist wherein components of the described system are leveraged with respect to additional domains. For example, the variational autoencoder and corresponding uncertainty quantification module(s) may be configured in additional embodiments to communicate with/receive data from any arrangement of sensors configured to provide sensor data. Further yet, the described system may be leveraged with respect to additional specific applications directed towards monitoring and analyzing brain activity. Certainly, many domains may benefit from the utilization of the described system for determining optimal sensor location placements for executing domain specific tasks. Notably, any system in which there are a limited number of sensors, spatially distributed time series signals, and a task to identify a latent state that depends for its success on an optimal placement of sensors could benefit from implementation of the systems and techniques described herein. The below additional applications and domains are intended to be illustrative and are not by any means exhaustive.

In at least some additional embodiments, the described system may be configured for neurofeedback treatment applications. In such embodiments, the variational autoencoder may be configured for optimizing a task, such as pattern identification within EEG data. Such a pattern may correspond to a pattern of functional connectivity, to a specific cognitive state, and the like. As such, identification of such a pattern can be useful for neurofeedback treatment that targets either activation of a pattern of functional connectivity or the presence of a cognitive state, for example. As such, the components of the described system can be configured to identify which sensor placement maximizes a probability of decoding a cognitive state, thereby increasing the reliability of neurofeedback treatments by increasing the probability of correctly identifying a subject cognitive state.

In at least some additional embodiments, the described system may be configured for pathological brain activity applications. A dataset used for training a decoder to predict a pattern of functional connectivity or cognitive state can be biased if it only relies on a limited subset of electrode positions. If the dataset relies on a limited subset of electrode positions, the selected subset may be insufficient to train a decoder as accurately as possible for another population. For instance, a dataset gathered from a healthy population using certain electrode positions may not be useful for training a decoder for a clinical population that exhibits a certain neurological disorder. The systems as described herein, therefore, can be leveraged to help improve electrode positions for said clinical population.

In at least some additional embodiments, the described system may be configured for precision medicine applications. Even if certain electrode positions have been shown to be optimal for decoding a pattern of functional connectivity or cognitive state for a certain population, such globally optimized electrode positions may not be optimal for each individual of that population. For example, there may be anatomical differences between individuals of the population that make a subset of electrodes and/or electrode placements optimal for some individuals and suboptimal for others. For example, skull shape and axonal connectivity can impact optimal position for an individual specifically such that an optimal position as determined for the entire population may not be optimal for the individual on the basis of these conditions/anatomical features. As such, for an electrode or other sensor to be optimized for the individual, it may need to be moved away from a globally optimized location. In such applications, the system may be configured to recommend an updated position responsive to the detection of such features.

### Conclusion

Many modifications and other embodiments will come to mind to one skilled in the art to which this disclosure pertains having the benefit of the teachings presented in the foregoing descriptions and the associated drawings. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

### Examples

Example 1. A computer-implemented method comprising: receiving, by one or more processors, electroencephalography (EEG) data and one or more current electrode positions from an EEG device; determining a subject EEG task corresponding to a current state of the EEG device; generating a latent space representation corresponding to the EEG data and the one or more current electrode positions; generating an uncertainty measure corresponding to the latent space representation and the EEG data; generating one or more preferred electrode positions corresponding to the subject EEG task and the EEG device; and generating a visual representation of the one or more preferred electrode positions.

Example 2. The computer-implemented method of Example 1, wherein generating the uncertainty measure comprises using a Bayesian algorithm.

Example 3. The computer-implemented method of any of the preceding Examples, further comprising causing display of the visual representation of the one or more preferred electrode positions.

Example 4. The computer-implemented method of any of the preceding Examples, further comprising refining the latent space representation for improved calibration by identifying areas requiring additional sampling.

Example 5. The computer-implemented method of any of the preceding Examples, further comprising predicting a brain state or brain features corresponding to a user of the EEG device based at least in part on the EEG data and the uncertainty measure.

Example 6. The computer-implemented method of any of the preceding examples, wherein the subject EEG task is emotional inference of a user of the EEG device.

Example 7. The computer-implemented method of any of the preceding examples, wherein the subject EEG task is motor intent of a user of the EEG device.

Example 8. A computing apparatus comprising memory and one or more processors communicatively coupled to the memory, the one or more processors configured to perform operations comprising: receiving electroencephalography (EEG) data and one or more current electrode positions from an EEG device; determining a subject EEG task corresponding to a current state of the EEG device; generating a latent space representation corresponding to the EEG data and the one or more current electrode positions; generating an uncertainty measure corresponding to the latent space representation and the EEG data; generating one or more preferred electrode positions corresponding to the subject EEG task and the EEG device; and generating a visual representation of the one or more preferred electrode positions.

Example 9. The computing apparatus of example 8, wherein generating the uncertainty measure comprises using a Bayesian algorithm.

Example 10. The computing apparatus of any of the preceding examples, the operations further comprising causing display of the visual representation of the one or more preferred electrode positions.

Example 11. The computing apparatus of any of the preceding examples, the operations further comprising refining the latent space representation for improved calibration by identifying areas requiring additional sampling.

Example 12. The computing apparatus of any of the preceding examples, the operations further comprising predicting a brain state or brain features corresponding to a user of the EEG device based at least in part on the EEG data and the uncertainty measure.

Example 13. The computing apparatus of any of the preceding examples, wherein the subject EEG task is emotional inference of a user of the EEG device.

Example 14. The computing apparatus of any of the preceding examples, wherein the subject EEG task is motor intent of a user of the EEG device.

Example 15. One or more non-transitory computer-readable storage media including instructions that, when executed by one or more processors, cause the one or more processors to perform operations comprising: receiving electroencephalography (EEG) data and one or more current electrode positions from an EEG device; determining a subject EEG task corresponding to a current state of the EEG device; generating a latent space representation corresponding to the EEG data and the one or more current electrode positions; generating an uncertainty measure corresponding to the latent space representation and the EEG data; generating one or more preferred electrode positions corresponding to the subject EEG task and the EEG device; and generating a visual representation of the one or more preferred electrode positions.

Example 16. The one or more non-transitory computer-readable storage media of example 15, wherein generating the uncertainty measure comprises using a Bayesian algorithm.

Example 17. The one or more non-transitory computer-readable storage media of any of the preceding examples, the operations further comprising causing display of the visual representation of the one or more preferred electrode positions.

Example 18. The one or more non-transitory computer-readable storage media of any of the preceding examples, the operations further comprising refining the latent space representation for improved calibration by identifying areas requiring additional sampling.

Example 19. The one or more non-transitory computer-readable storage media of any of the preceding examples, the operations further comprising predicting a brain state or brain features corresponding to a user of the EEG device based at least in part on the EEG data and the uncertainty measure.

Example 20. The one or more non-transitory computer-readable storage media of any of the preceding examples, wherein the subject EEG task is emotional inference of a user of the EEG device.

1. A computer-implemented method comprising:
receiving, by one or more processors, electroencephalography (EEG) data and one or more current electrode positions from an EEG device;
determining, by the one or more processors, a subject EEG task corresponding to a current state of the EEG device;
generating, by the one or more processors, a latent space representation corresponding to the EEG data and the one or more current electrode positions;
generating, by the one or more processors, an uncertainty measure corresponding to the latent space representation and the EEG data;
generating, by the one or more processors and based at least in part on the uncertainty measure and the subject EEG task, one or more preferred electrode positions corresponding to the subject EEG task and the EEG device; and
generating, by the one or more processors, a visual representation of the one or more preferred electrode positions.

2. The computer-implemented method of claim 1, wherein generating the uncertainty measure comprises using a Bayesian algorithm.

3. The computer-implemented method of claim 1 further comprising causing display of the visual representation of the one or more preferred electrode positions.

4. The computer-implemented method of claim 1, further comprising refining the latent space representation for improved calibration by identifying areas exhibiting insufficient sampling.

5. The computer-implemented method of claim 1, further comprising generating a prediction of a brain state or brain features corresponding to a user of the EEG device based at least in part on the EEG data and the uncertainty measure.

6. The computer-implemented method of claim 1, wherein the subject EEG task is emotional inference of a user of the EEG device.

7. The computer-implemented method of claim 1, wherein the subject EEG task is motor intent of a user of the EEG device.

8. A computing apparatus comprising memory and one or more processors communicatively coupled to the memory, the one or more processors configured to perform operations comprising:
receiving electroencephalography (EEG) data and one or more current electrode positions from an EEG device;
determining a subject EEG task corresponding to a current state of the EEG device;
generating a latent space representation corresponding to the EEG data and the one or more current electrode positions;
generating an uncertainty measure corresponding to the latent space representation and the EEG data;
generating, based at least in part on the uncertainty measure and the subject EEG task, one or more preferred electrode positions; and
generating a visual representation of the one or more preferred electrode positions.

9. The computing apparatus of claim 8, wherein generating the uncertainty measure comprises using a Bayesian algorithm.

10. The computing apparatus of claim 8, the operations further comprising causing display of the visual representation of the one or more preferred electrode positions.

11. The computing apparatus of claim 8, the operations further comprising refining the latent space representation for improved calibration by identifying areas exhibiting insufficient sampling.

12. The computing apparatus of claim 8, the operations further comprising generating a prediction of a brain state or brain features corresponding to a user of the EEG device based at least in part on the EEG data and the uncertainty measure.

13. The computing apparatus of claim 8, wherein the subject EEG task is emotional inference of a user of the EEG device.

14. The computing apparatus of claim 8, wherein the subject EEG task is motor intent of a user of the EEG device.

15. One or more non-transitory computer-readable storage media including instructions that, when executed by one or more processors, cause the one or more processors to perform operations comprising:
receiving electroencephalography (EEG) data and one or more current electrode positions from an EEG device;
determining a subject EEG task corresponding to a current state of the EEG device;
generating a latent space representation corresponding to the EEG data and the one or more current electrode positions;
generating an uncertainty measure corresponding to the latent space representation and the EEG data;
generating, based at least in part on the uncertainty measure and the subject EEG task, one or more preferred electrode positions; and
generating a visual representation of the one or more preferred electrode positions.

16. The one or more non-transitory computer-readable storage media of claim 15, wherein generating the uncertainty measure comprises using a Bayesian algorithm.

17. The one or more non-transitory computer-readable storage media of claim 15, the operations further comprising causing display of the visual representation of the one or more preferred electrode positions.

18. The one or more non-transitory computer-readable storage media of claim 15, the operations further comprising refining the latent space representation for improved calibration by identifying areas exhibiting insufficient sampling.

19. The one or more non-transitory computer-readable storage media of claim 15, the operations further comprising generating a prediction of a brain state or brain features corresponding to a user of the EEG device based at least in part on the EEG data and the uncertainty measure.

20. The one or more non-transitory computer-readable storage media of claim 15, wherein the subject EEG task is emotional inference of a user of the EEG device.
